(19) United States
(12) Patent Application Publication
(10) Pub. No.: US 2024/0262706 A1
(43) Pub. Date: Aug. 8, 2024

(54) RECYCLING OF SCRAP DENTAL ZIRCONIA BLOCK

(71) Applicant: James R. Glidewell Dental Ceramics, Inc., Newport Beach, CA (US)

(72) Inventors: Dongkyu Kim, Irvine, CA (US); Sreeram Balasubramanian, Irvine, CA (US); Hugo Gonzalez, Whittier, CA (US); Mathew Campbell, Tustin, CA (US); Rajashekharam Malyala, Camarillo, CA (US)

(73) Assignee: James R. Glidewell Dental Ceramics, Inc., Newport Beach, CA (US)

(21) Appl. No.: 18/428,929

(22) Filed: Jan. 31, 2024

Related U.S. Application Data
(60) Provisional application No. 63/483,062, filed on Feb. 3, 2023.

(51) Int. Cl. C01G 25/02; A61C 13/00; A61K 6/818; C09C 3/04

(52) U.S. Cl. CPC C01G 25/02; A61C 13/0004; A61C 13/0022; A61K 6/818; C09C 3/041; A61C 2201/00; C01P 2004/03; C01P 2004/61; C01P 2004/62; C01P 2004/64

(57) ABSTRACT

A method that includes collecting scrap ZrO₂ material; reducing sizes of the scrap ZrO₂ material to produce a reduced-size scrap ZrO₂ material; preparing a ceramic slurry comprising the reduced-size scrap ZrO₂ material, deionized water, and at least one dispersant; milling the ceramic slurry to produce a milled ZrO₂ slurry, wherein the milled ZrO₂ slurry comprises a ZrO₂ powder with a particle size distribution D₍₅₀₎ of 0.05 μm to 1.0 μm; and casting the milled ZrO₂ slurry to prepare a ceramic block.

This application claims the benefit of U.S. Provisional Patent Appl. No. 63/483,062, filed Feb. 3, 2023, which is incorporated herein by reference in its entirety.

### FIELD

Methods for preparing a zirconia powder from scrap zirconia blocks, and methods for making a ceramic block using the prepared zirconia powder are disclosed.

### BACKGROUND

About 43% of CAD/CAM ZrO₂ materials are wasted after milling restorations in the dental industry. Existing industrial processes for recycling the wasted ZrO₂ materials do not produce end products with desirable characteristics comparable to commercial products.

A significant technological gap exists in the art regarding making commercial quality ZrO₂ powders that can be used to make blocks from which dental restorations could be milled, wherein the properties of restorations need to be comparable to those of restorations made by commercial grade powder.

### SUMMARY

Disclosed herein are methods that include:
collecting scrap ZrO₂ material;
reducing sizes of the scrap ZrO₂ material to produce a reduced-size scrap ZrO₂ material;
preparing a ceramic slurry comprising the reduced-size scrap ZrO₂ material, deionized water, and at least one dispersant;
milling the ceramic slurry to produce a milled ZrO₂ slurry, wherein the milled ZrO₂ slurry comprises a ZrO₂ powder with a particle size distribution D₍₅₀₎ of 0.05 μm to 1.0 μm; and casting the milled ZrO₂ slurry to prepare a ceramic block.

The foregoing will become more apparent from the following detailed description, which proceeds with reference to the accompanying figures.

### BRIEF DESCRIPTION OF THE DRAWINGS

FIGS. 1A-1D show SEM micrographs of as received, commercial Tosoh PX-328 powder and different scrap ZrO₂ powders after size reduction and sieving. FIG. 1A shows as received Tosoh PX-328 powder; FIG. 1B shows recycled ZrO₂ powder with particle size of 75˜150 μm; FIG. 1C shows recycled ZrO₂ powder with particle size of 38˜75 μm; FIG. 1D shows recycled ZrO₂ powder with particle size smaller than 38 μm.

FIG. 2 shows particle size distribution of recycled ZrO₂ powder after attrition milling for 1 hour using Netzsch mill.

FIGS. 3A-3D show SEM micrographs of recycled powders attrition milled for 1 hour using Netzsch mill at ×50 (FIG. 3A), ×1000 (FIG. 3B), ×5000 (FIG. 3C), and ×20000 (FIG. 3D).

FIGS. 4A-4H show SEM micrograph of as received, new, Tosoh powder and those of attrition milled, dried, ball milled, and then sieved powders with different size groups. FIGS. 4A-4D show SEM micrograph at ×200 magnification of PX328 powder (FIG. 4A), 75˜150 μm powder (FIG. 4B), 38˜75 μm powder (FIG. 4C), and ≤38 μm powder (FIG. 4D); FIGS. 4E-4H show SEM micrograph at ×20,000 magnification of PX328 powder (FIG. 4E), 75˜150 μm powder (FIG. 4F), 38˜75 μm powder (FIG. 4G), and ≤38 μm powder (FIG. 4H).

FIGS. 5A-5D show SEM micrographs of sintered recycled ZrO₂ disks which were pressed by using powders with size of 75˜150 μm and at a pressure of 35.8 ksi (FIG. 5A), powders with size of 38˜75 μm and at pressure of 35.8 ksi (FIG. 5B), powders with size of 38˜75 μm and at a pressure of 57.4 ksi (FIG. 5C), powders with size of ≤38 μm and at a pressure of 35.8 ksi (FIG. 5D).

FIGS. 6A-6C show optical micrographs of 3 different types of scrap ZrO₂ materials. FIG. 6A shows pre-size reduced scrap ZrO₂ materials with size smaller than 4.75 mm; FIG. 6B shows scrap ZrO₂ blocks with thicknesses of 14 mm, with diameters of 98 mm, and with minimal remaining ZrO₂; FIG. 6C shows scrap ZrO₂ blocks with thickness of 20 mm, with diameter of 98 mm, and with larger quantity remaining ZrO₂.

FIGS. 7A-7B show optical micrographs of final remaining particles larger than 100 μm after wet ball milling for 10 hour, from scrap ZrO₂ blocks with thicknesses of 14 mm, with diameters of 98 mm, and with minimal remaining ZrO₂ (FIG. 7A), scrap ZrO₂ blocks with thicknesses of 20 mm, with diameters of 98 mm, and with larger quantity remaining ZrO₂ (FIG. 7B).

FIG. 8 shows recycled, green, 102 mm diameter block pressed at 24.4 ksi. Density of the block is 3.22 g/cm³ (52.7%).

FIG. 9 shows two different groups of 3 unit bridges and single crown. Top: milled from commercial BruxZir shaded 16(+) A2 shade block, Bottom: milled from recycled BruxZir shaded 16(+) A2 shade block.

FIGS. 10A-10B show SEM micrographs of two different ZrO₂ blocks. FIG. 10A shows ZrO₂ blocks made from commercial shaded 16(+) A2 shade powder; FIG. 10B shows ZrO₂ blocks made from recycled shaded 16(+) A2 shade powder.

FIGS. 11A-11C show optical micrographs showing insides of chambers of ball mill after 10 hour milling by using 5 mm diameter spherical balls (FIG. 11A), by using 20 mm diameter cylindrical balls (FIG. 11B), and by using mixed 5 mm diameter spherical balls and 20 mm diameter cylindrical balls (FIG. 11C).

FIG. 12A-12B show particle size distribution after ball milling for 10 hour using cylindrical 20 mm diameter cylindrical ZrO₂ balls (FIG. 12A) and mixed 5 mm diameter spherical ZrO₂ balls and 20 mm diameter cylindrical ZrO₂ balls (FIG. 12B).

FIGS. 13A-13C show optical micrographs showing inside of ball mill container after ball milling scrap ZrO₂ particles with size of smaller than 4.75 mm (4750 μm) by using cylindrical ZrO₂ balls with diameter 20 mm and height of 20 mm. FIG. 13A shows results after milling for 3 hour; FIG. 13B shows results after milling for 5 hour; and FIG. 13C shows results after milling for 10 hour.

FIGS. 14A-14C show particle size distribution after ball milling scrap ZrO₂ particles with size of smaller than 4.75 mm (4750 μm) by using cylindrical ZrO₂ balls with diameter 20 mm and height of 20 mm. FIG. 14A shows results after milling for 3 hour, D50 is 0.300 μm; FIG. 14B shows results after milling for 5 hour, D50 is 0.272 μm; and FIG. 14C shows results after milling for 10 hour, D50 is 0.243 μm.

FIG. 15A-15B show optical micrographs showing casted blocks after drying for 3 days. FIG. 15A shows all blocks which casted after 3 hour ball milling were delaminated; FIG. 15B shows 3 blocks out of 5 blocks which casted after 5 hour ball milling were delaminated.

FIG. 16A-16B show optical micrographs showing inside of ball mill container after ball milling pre-milled scrap ZrO₂ particles with size of smaller than 4.75 mm (4750 μm) by using cylindrical balls with diameter of 20 mm and height of 20 mm. FIG. 16A shows results after wet milling for 10 hour; and FIG. 16B shows results after dry milling for 10 hour.

FIG. 17A-17B show optical micrographs showing inside of ball mill container after ball milling of pre-milled scrap ZrO₂ particles smaller than 4.75 mm (4750 μm) by using mixture of spherical balls with diameter of 5 mm and cylindrical balls with diameter of 20 mm and height of 20 mm. FIG. 17A shows results after wet ball milling for 10 hour; and FIG. 17B shows results after dry ball milling for 10 hour.

FIGS. 18A-18B show optical micrographs showing inside of ball mill chamber after ball milling for 5 hour (FIG. 18A), and 10 hour (FIG. 18B).

FIGS. 19A-19C show optical micrographs of ZrO₂ blocks made from scrap ZrO₂ powders obtained after casting of scrap ZrO₂ slurry made by ball milling scrap ZrO₂ powder for 5 hour (FIG. 19A), ball milling scrap ZrO₂ powder for 10 hour (FIG. 19B), ball milling for 10 hour and attrition milling for 1 hour (FIG. 19C).

FIGS. 20A-20C show SEM micrographs of blocks made from powders of commercial shaded 16(+) A2 powder (FIG. 20A), recycled shaded 16(+) A2 powder after ball milling for 10 hour (FIG. 20B), recycled shaded 16(+) A2 powder after ball milling for 10 hour and then Netzsch milling for 1 hour (×2000) (FIG. 20C).

FIGS. 21A-21C show SEM micrographs of blocks made from powders of commercial shaded 16(+) A2 powder (FIG. 21A), recycled shaded 16(+) A2 powder after ball milling for 10 hour (FIG. 21B), and recycled shaded 16(+) A2 powder after ball milling for 10 hour and then Netzsch milling for 1 hour (×5,000) (FIG. 21C).

FIGS. 22A-22C show SEM micrographs of blocks made from powders of commercial shaded 16(+) A2 powder (FIG. 22A), recycled shaded 16(+) A2 powder after ball milling for 10 hour (FIG. 22B), recycled shaded 16(+) A2 powder after ball milling for 10 hour and then Netzsch milling for 1 hour (×10,000) (FIG. 22C).

FIG. 23 shows sintered 3-unit bridge and single crown milled (top) from new BruxZir shaded 16(+) A2 shade block and (bottom) from recycled BruxZir shaded 16(+) A2 shade block.

### DETAILED DESCRIPTION

This disclosure concerns embodiments of a method for preparing a ZrO₂ powder from scrap ZrO₂ blocks, as well as embodiments of a method for preparing a ceramic block using the ZrO₂ powder.

As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximates unless the word “about” is recited.

### Preparing a ZrO₂ Powder

In the methods disclosed herein scrap dental ZrO₂ material is recycled to make dental grade ZrO₂ powder that can be used to make dental ZrO₂ blocks from which dental prosthetic devices can be milled. In some embodiments, the scrap dental ZrO₂ material is waste material resulting from milling of new or fresh ZrO₂ blocks.

Methods of preparing a ZrO₂ powder from scrap ZrO₂ materials are disclosed. The methods involve controlled size reduction that reduces the powder into submicron size, forming methods that controls agglomeration, and processing control methods such as addition of binders. The properties of prosthetic devices milled from a block made by recycled ZrO₂ powder were comparable to those of prosthetic devices milled from a block made by new ZrO₂ powder.

The method includes milling the scrap ZrO₂ materials to produce a milled ZrO₂ slurry. In some embodiments, the scrap ZrO₂ materials are directly milled into milled ZrO₂ slurry without preceding size reduction steps. In some other embodiments, the method includes applying preceding size reduction steps to the scrap ZrO₂ materials before the final milling step to obtain milled ZrO₂ slurry. In certain embodiments, the preceding size reduction steps include reducing sizes of the scrap ZrO₂ materials to produce a reduced-size scrap ZrO₂ material, and sieving the ZrO₂ material to produce a sieved scrap ZrO₂. In one embodiment, the preceding size reduction steps include collecting scrap ZrO₂ material (e.g., the portion of a block remaining after dental prosthetic devices were formed from the block), reducing sizes of the scrap ZrO₂ material to produce a reduced-size scrap ZrO₂ material, and sieving the reduced-size ZrO₂ to produce a sieved, reduced-size ZrO₂ material.

In certain embodiments, the milled ZrO₂ slurry comprises ZrO₂ powder with a particle size distribution D₍₅₀₎ of 0.05 μm to 1.0 μm, or 0.07 μm to 0.8 μm, or 0.08 μm to 0.7 μm, or 0.1 μm to 0.5 μm. As used herein, the term “particle size” refers to measurements obtained by a Brookhaven Instruments Corp. X-ray disk centrifuge analyzer.

In some other embodiments, the method includes collecting scrap ZrO₂ material, reducing sizes of the scrap ZrO₂ material to produce a reduced-size scrap ZrO₂ material, sieving the reduced-size ZrO₂ material to produce a sieved, reduced-size ZrO₂ material, and wet milling the sieved, reduced-size ZrO₂ material to produce a milled ZrO₂ slurry.

### Scrap ZrO₂ Materials

The scrap ZrO₂ materials may be from stabilized zirconia ceramic material that includes both fully and partially stabilized zirconia. Zirconia may be stabilized with approximately 1 mol % to approximately 8 mol % yttria, or approximately 2 mol % to approximately 6 mol % yttria, or approximately 2 mol % to approximately 6.5 mol % yttria, or approximately 2 mol % to approximately 5.5 mol % yttria, or approximately 2 mol % to approximately 5 mol % yttria, or approximately 2 mol % to approximately 4 mol % yttria. Yttria-stabilized zirconia ceramic scrap materials may comprise up to 7.5 mol % yttria, or up to 8.5 mol % yttria, for example, from 5 mol % yttria to 8.5 mol % yttria, from 5 mol % yttria to 8 mol % yttria, from 5 mol % yttria to 7.5 mol % yttria, 5 mol % yttria to 6.4 mol % yttria, from 5 mol % yttria to 5.6 mol % yttria, from 5.1 mol % yttria to 6.4 mol % yttria, from 5.2 mol % yttria to 7.5 mol % yttria, from 5.2 mol % yttria to 7.0 mol % yttria, from 5.4 mol % yttria to 7.5 mol % yttria, from 5.4 mol % yttria to 7.0 mol % yttria, from 5.5 mol % yttria to 7.5 mol % yttria, from 5.5 mol % yttria to 7 mol % yttria, from 5.5 mol % yttria to 6.9 mol % yttria, from 5.5 mol % yttria to 6 mol % yttria, from 5.5 mol % yttria to 5.9 mol % yttria, from 5.6 mol % yttria to 6.3 mol % yttria, from 5.7 mol % yttria to 6.3 mol % yttria, from 5.8 mol % yttria to 6.3 mol % yttria, from 6 mol % yttria to 8.5 mol % yttria, from 6 mol % yttria to 8 mol % yttria, from 6.0 mol % yttria to 7.5 mol % yttria, from 6 mol % yttria to 7 mol % yttria, from 6.0 mol % yttria to 6.8 mol % yttria, from 6.0 mol % yttria to 6.3 mol % yttria, from 6.2 mol % yttria to 7.5 mol % yttria, from 6.4 mol % yttria to 7.5 mol % yttria, from 7 mol % yttria to 8.5 mol % yttria, or from 7.2 mol % to 8.4 mol % yttria, to stabilize zirconia.

In some embodiments, the scrap ZrO₂ material comprise pre-size reduced scrap ZrO₂ materials with a largest dimension size smaller than 10.0 mm, smaller than 5.00 mm, or smaller than 1.00 mm.

In some other embodiments, the scrap ZrO₂ material comprise scrap ZrO₂ blocks with thicknesses of 14 mm, with diameters of 98 mm, and with 10 wt % to less than 90 wt % remaining ZrO₂, or 20 wt % to less than 70 wt % remaining ZrO₂, or 30 wt % to less than 50 wt % remaining ZrO₂.

In some embodiments, the scrap ZrO₂ material comprise scrap ZrO₂ blocks with thickness of 20 mm, with diameter of 98 mm, and with 10 wt % to less than 90 wt % remaining ZrO₂, or 20 wt % to less than 70 wt % remaining ZrO₂, or 30 wt % to less than 50 wt % remaining ZrO₂.

### Size Reduction

In some embodiments, the sizes of the scrap ZrO₂ material are reduced to produce a reduced-size scrap ZrO₂ material. In certain embodiments, the size reduction can occur via a mechanical process, such as mechanical milling. Mechanical milling can be conducted in dry or wet condition. Size reduction can be happened by using different mechanical milling techniques such as jaw crusher, impact crusher, impact mill, roller mill, ball mill, jet mill, Netzsch mill, or agitated media mill.

In some embodiments, the reduced-size scrap ZrO₂ material comprises ZrO₂ particles with a particle size distribution D₍₅₀₎ of 1 mm to 10 mm.

### Sieving

In some embodiments, the scrap ZrO₂ material is sieved to produce a sieved ZrO₂ material. In certain embodiments, the scrap ZrO₂ material is sieved through a plurality of sieves with opening dimeters ranging from 1.3 μm to 8000 μm.

In some embodiments, the sieved ZrO₂ material comprises ZrO₂ particles with particle sizes of 0.0013 mm to 8 mm, or 0.018 mm to 1 mm, or 0.038 mm to 0.160 mm. In certain embodiments, the sieved ZrO₂ material comprises ZrO₂ particles with particle sizes of less than 4.75 mm.

### Slurry Formation

The reduced-size ZrO₂ scrap material (or the sieved, reduced-size scrap ZrO₂ material) is mixed with deionized water and at least one dispersant to form a scrap ZrO₂ slurry. In certain embodiments, the scrap ZrO₂ slurry comprises 30 wt % to 85 wt %, more particularly 50 wt % to 75 wt %, scrap ZrO₂ material, 15 wt % to 70 wt %, more particularly 25 wt % to 50 wt %, deionized water, and 0.1 wt % to 7 wt %, more particularly 0.2 wt % to 3 wt %, dispersant to the weight of ZrO₂ material, based on the total weight of the slurry.

Dispersants used to form ceramic suspensions or ceramic slurries to form green bodies by slip-casting manufacturing methods such as those described herein, function by promoting the dispersion and/or stability of the slurry and/or decreasing the viscosity of the slurry. Dispersion and deagglomeration may occur through electrostatic, electrosteric, or steric stabilization. Examples of suitable dispersants include nitric acid, hydrochloric acid, citric acid, diammonium citrate, triammonium citrate, polycitrate, polyethyleneimine, polyacrylic acid, polymethacrylic acid, polymethacrylate, polyethylene glycols, polyvinyl alcohol, polyvinyl pyrillidone, carbonic acid, and various polymers and salts thereof. These materials may be either purchased commercially, or prepared by known techniques. Specific examples of commercially available dispersants include Darvan® 821-A ammonium polyacrylate dispersing agent commercially available from Vanderbilt Minerals, LLC; Dolapix™ CE 64 organic dispersing agent and Dolapix™ PC 75 synthetic polyelectrolyte dispersing agent commercially available from Zschimmer & Schwarz GmbH; and Duramax™ D 3005 ceramic dispersant commercially available from Dow Chemical Company.

The scrap ZrO₂ slurry is subjected to milling.

In some embodiments, the milling process is dry milling. In some embodiments, the milling process is dry ball milling. In some embodiments, the milling process is wet milling. In certain embodiments, the milling process is wet ball milling.

In some embodiments, the milling process is wet ball milling using balls with diameters ranging from 0.5 mm to 50 mm. In certain embodiments, the wet ball milling comprises using balls with diameters ranging from 5 mm to 20 mm. In one embodiment, the wet ball milling comprises using a mixture of 5 mm and 20 mm diameter cylindrical balls.

In some embodiments, the wet milling is performed for a certain period of time. For example, the slurry may be wet milled for 0.5 hours to 48 hours, or 3 hours to 15 hours. In one example, the slurry may be wet milled for 10 hours.

In certain embodiments, the ZrO₂ powders are wet ball milled with at least one binder (e.g., a polyvinyl alcohol). In one example, the binder may be added in an amount ranging from 0.1 wt % to 10 wt %, based on the total weight of the slurry. In another example, the PVA binders were added in an amount of 2 wt %, based on the total weight of the slurry.

In some embodiments, the ZrO₂ slurry after wet milling comprises a ZrO₂ powder with a particle size distribution D₍₅₀₎ of 0.05 μm to 5 μm, or 0.1 μm to 1 μm, or 0.15 μm to 0.5 μm.

### Attrition Milling

The post-wet milled ZrO₂ slurry may be attrition milled. For example, via Netzsch milling. The attrition milling is performed for a certain time period. For example, the slurry may be attrition milled for 0.1 hours to 10 hours, or 0.5 hours to 3 hours. In one example, the slurry may be attrition milled for 1 hour.

In some embodiments, the ZrO₂ slurry after attrition milling comprises a ZrO₂ powder with a particle size distribution D₍₅₀₎ of 0.05 μm to 3 μm, or 0.07 μm to 0.5 μm, or 0.1 μm to 0.3 μm.

### Preparing ZrO₂ Blocks

In further embodiments, the milled ZrO₂ slurry is casted to prepare a ceramic block. In certain embodiments, the casting technique is slip casting. In one embodiment, the slip casting is an improved slip-casting process provided by U.S. Pat. Nos. 10,532,008, 9,434,651, and U.S. Patent Publication No. 2009/0115084, each of which is incorporated herein by reference in its entirety. In another embodiment, pressure casting may be used to prepare a ceramic block. In a further embodiment, dual vacuum-pressure casting may be used to prepare a ceramic block.

Alternatively, the milled ZrO₂ slurry may be spray dried to produce ZrO₂ powders. In further embodiments, the spray dried ZrO₂ powders are pressed to produce ceramic blocks.

Zirconia ceramic slurries may be cast into a desired shape, such as a solid block, disk, near net shape, or other shape. Ceramic slurries may be poured into a porous mold (e.g., plaster of Paris or other porous/filtration media) having the desired shape, and cast, for example, under the force of capillary action, vacuum, pressure, or a combination thereof (for example, by methods provided in US 2013/0313738, which is hereby incorporated by reference in its entirety). Green bodies may form a desired shape as water contained in the slurry is absorbed/filtered through the porous media. Excess slurry material, if any remaining, may be poured off the green body. Green bodies removed from molds may dry, for example, at room temperature in a humidity-controlled environment. Dental milling blanks may be cast, for example, as a solid block, disk, or near-net-shape, having dimensions suitable for use in milling or grinding single unit or multi-unit restorations, such as crowns, veneers, bridges, partial or full-arch dentures, and the like.

Manufacturing processes described herein may provide green bodies having relative densities ρ_{R} greater than 48%, such as from 52% to 65% relative density, or such as from 56% to 62% relative density. As used herein, the term “relative density” (ρ_{R}) refers to the ratio of the measured density ρ_{M} of a sample (g/cm³) to the theoretical density ρ_{T} (3 YSZ−6.083 g/mL; 5 YSZ−6.037 g/mL; 7 YSZ−5.991 g/mL).

Green bodies may be partially consolidated to obtain bisqued bodies by a heating step. Bisquing methods include heating or firing green bodies, such as green bodies in the shape of blocks to obtain, for example, porous bisqued blocks. In some embodiments, relative densities of bisque blocks do not increase more than 5% over the green body density. In some embodiments, the ceramic bodies are bisque heated so that the difference between the relative densities of the bisque body and the green body is 3% or less. Resulting bisqued bodies may be fully dried and have strength sufficient to withstand packaging, shipping, and milling, and in some embodiments, have a hardness value of less than or equal to 0.9 GPa, when tested by the hardness test method described herein. Bisque firing steps may include heating the green body at an oven temperature of from 800° C. to 1100° C. for a holding period of about 0.25 hours to 3 hours, or about 0.25 hours to 24 hours, or by other known bisquing techniques. In some embodiments, bisque processes comprise heating green bodies in an oven heated at an oven temperature of 900° C. to 1000° C. for 30 minutes to 5 hours.

Processes described herein may provide a bisqued body having a relative density ρ_{R} greater than or equal to 48%, such as from 48% to 62%, or from 54% to 60% Bisqued bodies may have a porosity of less than or equal to 45%, such as from 35% to 45%, or from 38% to 42%, or from 38% to 41%. As used herein, the term “porosity”, expressed as percent porosity above, is calculated as: percent porosity=1−percent relative density. A dental block for producing a dental prosthesis includes a zirconia bisqued body having a density of between 56% to 65% of theoretical density and having a porosity of between 35% and 44%, such as between 38% and 41%.

In some embodiments, the median pore size of bisque bodies is less than 200 nm, or less than 150 nm, less than 100 nm, such as from 30 nm to 150 nm, or from 30 nm to 80 nm, or from 35 nm to 40 nm, or from 40 nm to 80 nm, or from 40 nm to 70 nm, or from 45 nm to 75 nm, or from 45 nm to 50 nm, or from 50 nm to 80 nm, or from 50 nm to 75 nm, or from 55 nm to 80 nm, or from 55 nm to 75 nm, when measured according to the methods described herein. As used herein, the term “median pore diameter” refers to the pore diameter measurements obtained from a bisqued body via mercury intrusion performed with an Autopore V porosimeter from Micromeritics Instrument Corp.

Conventional subtractive processes, such as milling or machining processes known to those skilled in the art, may be used to shape a bisqued zirconia ceramic body or milling block into a pre-sintered dental restoration. For dental applications, a pre-sintered restoration may include a dental restoration such as a crown, a multi-unit bridge, an inlay or onlay, a veneer, a full or partial denture, or other dental restoration. For example, bisque stage blocks milled to form bisque-stage dental restorations having anatomical facial surface features including an incisal edge or biting surface, anatomical dental grooves and cusps, and are sintered to densify the bisque-stage restoration into the final dental restoration that may permanently installed in the mouth of a patient. In alternative embodiments, bisque-stage zirconia ceramic bodies are shaped into near-net-shape blocks having generic sizes and shapes that are sintered to theoretical density prior to machining into a final patient-specific dental restoration. The sintered near-net-shape bodies may be prepared having a shape and/or size that is suitable for range of similarly sized and shaped final restoration products.

Dental prostheses may be shaped from porous, pre-sintered blocks by conventional subtractive processes, such as milling or machining processes known to those skilled in the art. The blocks may be shaped in a crown, a multi-unit bridge, an inlay or onlay, a veneer, a full or partial denture, or other dental prosthesis.

In certain embodiments, the ceramic block is sintered to prepare a sintered ceramic block. In some embodiments, the ceramic block is sintered at a sintering temperature ranging from 1300° C. to 1650° C., or 1400° C. to 1600° C., or 1500° C. to 1550° C.

In some embodiments, the ceramic block is sintered for a time ranging from 0.5 hour to 15 hours, or 1 hour to 10 hours, or 2 hours to 7 hours.

### Properties of the ZrO₂ Blocks

In any of the foregoing or following embodiments, the sintered ceramic blocks produced by the disclosed methods have a flexural strength of 700 MPa to 1500 MPa, or 800 MPa to 1300 MPa, or 900 MPa to 1200 MPa.

In some embodiments, the sintered ceramic blocks produced by the disclosed methods have a fracture toughness of 1 MPa·m^{1/2} to 8 MPa·m^{1/2}, or 2 MPa·m^{1/2} to 7 MPa·m^{1/2}, or 3 MPa·m^{1/2} to 5 MPa·m^{1/2}.

In some embodiments, the sintered ceramic blocks produced by the disclosed methods have a transmittance of 30% to 65%, or 35% to 60% or 40% to 55% at 700 nm (when measured on a 1 mm thick fully sintered ceramic body).

### EXAMPLES

### Comparative Example 1

In this example, scrap CAD/CAM ZrO₂ blocks were mechanically broken down into smaller sizes and the broken particles were passed sequentially through 3 different sieves with opening diameters of 4750, 1700, and 500 μm. All the powders that passed through a sieve with an opening diameter of 500 μm were further size reduced by using a spin mixer to pass through a sieve with opening diameter of 150 μm. The final powders were sieved through stack of 3 sieves to classify powders into 3 different size groups of 75˜ 150 μm, 38˜75 μm, and ≤38 μm.

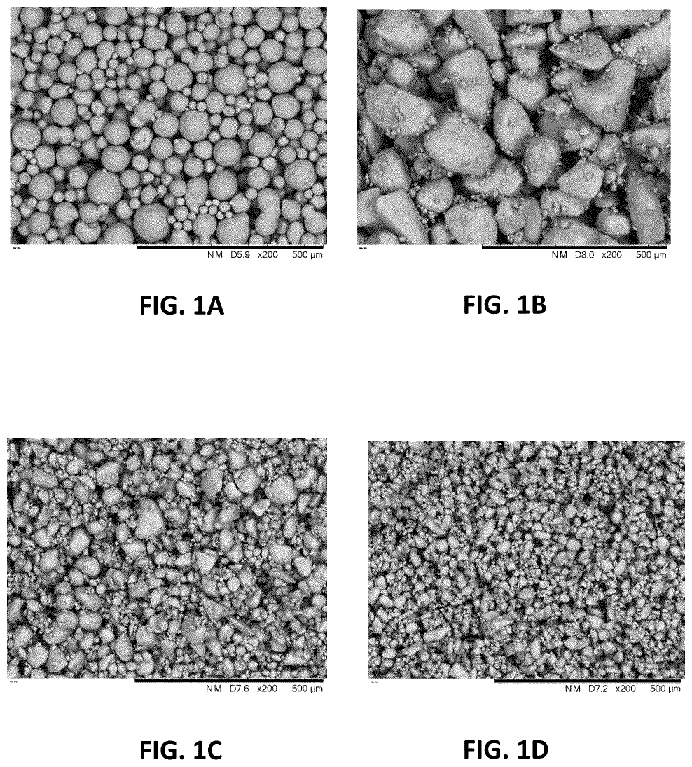

The morphologies of as received, new, Tosoh PX-328 powder and 3 different scrap ZrO₂ powders with different sizes are shown in FIGS. 1A-1D.

3 different recycled powders with different sizes were pressed into disks with diameter of 20 mm using a Carver press at pressures of 24.4 ksi, 35.8 ksi, and 57.4 ksi. However, in this example, as shown in Table 1, all the disk samples were delaminated and could not be successfully pressed.

TABLE 1
Pressing of different, recycled, scrap, ZrO₂ powders with different
particle sizes by using different pressing pressures.

Pressing pressure

| | | | |
|---|---|---|---|
| Particle size | 24.4 ksi | 35.8 ksi | 57.4 ksi |

| | | | | |
|---|---|---|---|---|
| 75~150 | μm | Delaminated | Delaminated | Delaminated |
| 38~75 | μm | Delaminated | Delaminated | Delaminated |
| ≤38 | μm | Delaminated | Delaminated | Delaminated |

### Example 2

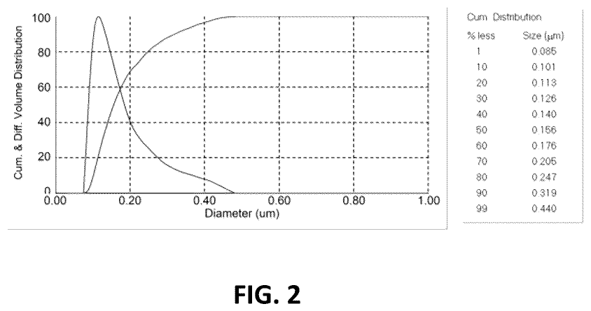

In this example, scrap ZrO₂ blocks were mechanically size reduced, and the broken particles were passed sequentially through 3 different sieves with opening diameters of 4750, 1700, and 500 μm. All of the powders that passed through a sieve with opening diameter of 500 μm were further size reduced by using a spin mixer to pass through a sieve with size of 150 μm. The final powders were sieved through stack of 3 sieves to classify powders into 3 different size groups of 75˜ 150 μm, 38˜75 μm, and ≤38 μm. A total of 2000 g of powder which was composed of 1720 g of powder with size of 38˜ 75 μm and 280 g of powder with size of ≤38 μm was mixed with 778 g of DI water and 10 g of Dolapix CE-64 as a dispersant. The mixture was attrition milled by using a Netzsch mill at 2500 rpm for 1 hour. After milling for 1 hour, the d50 of powder was 0.156 μm as shown in FIG. 2.

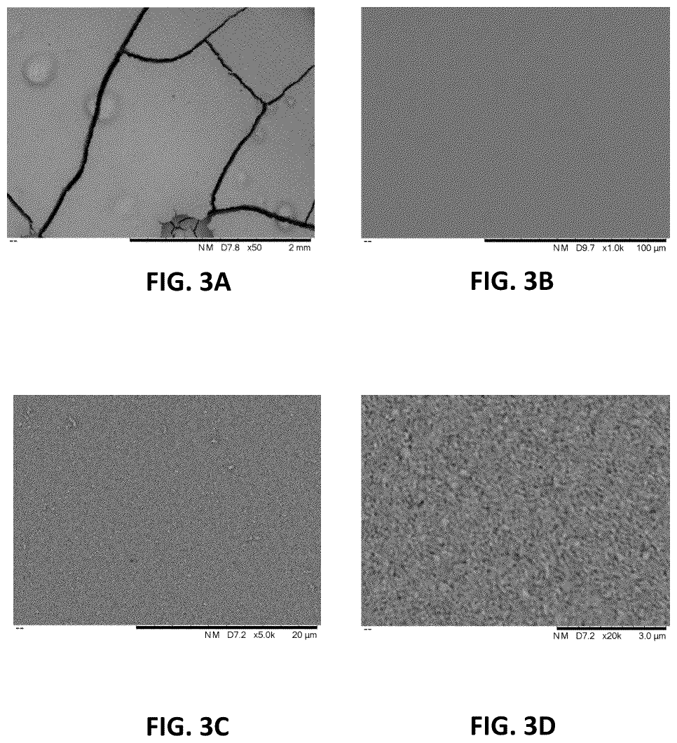

After attrition milling by Netzsch mill, the powder was dried at 110° C. for 2 hours, the morphologies of the powder were observed by SEM, and the micrographs are presented in FIG. 3. The powder showed very fine morphologies.

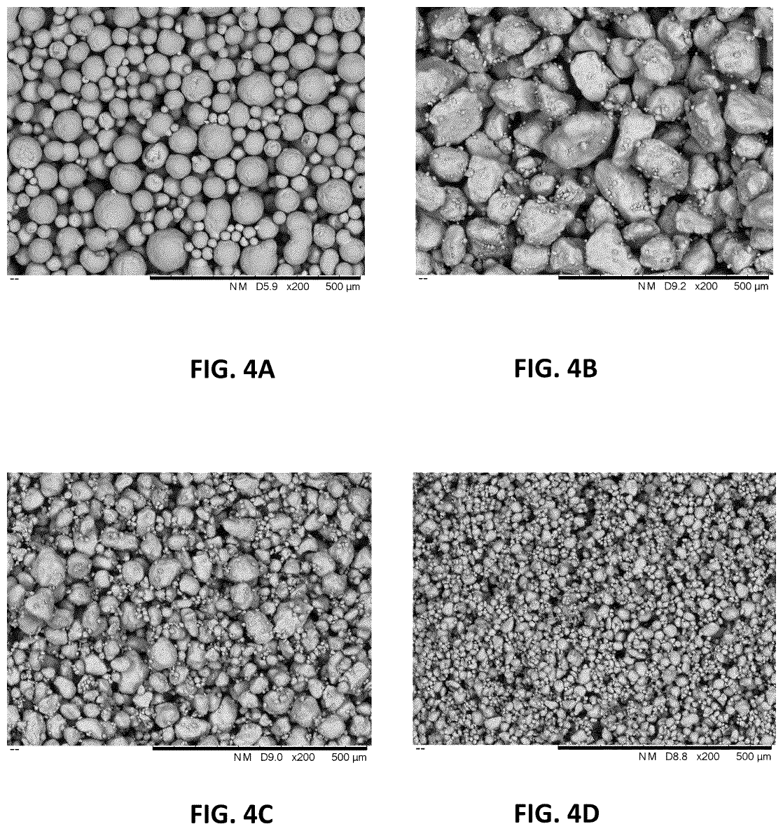

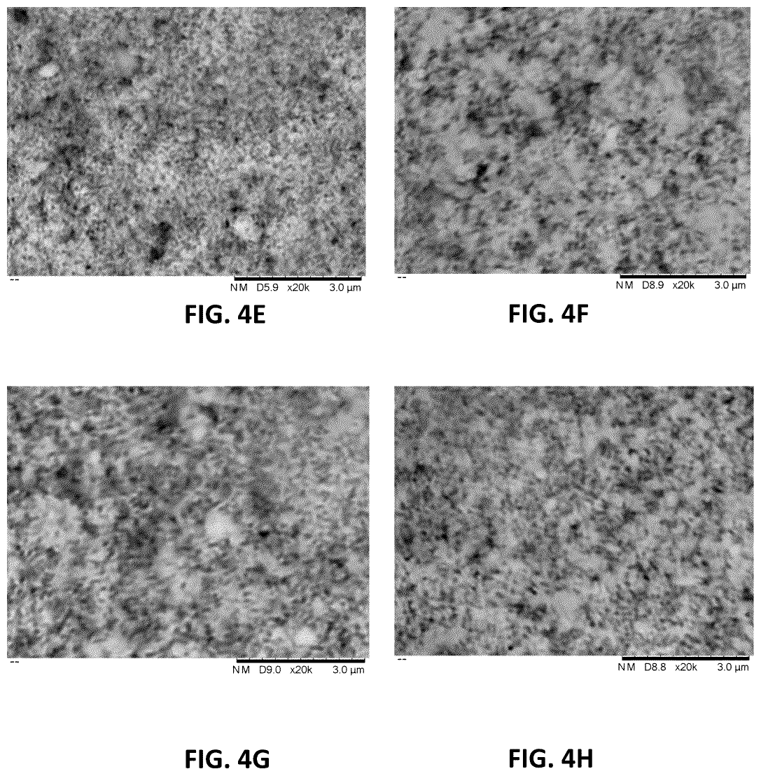

After attrition milling by Netzsch mill for 1 hour and drying for 1 hour at 110° C., the powder was re-agglomerated. To break down the agglomeration, the re-agglomerated powder was milled in the Spex mixer with 150 g of 2 mm diameter spherical ZrO₂ balls for 1 min followed by milling with 10 g of 1 mm diameter spherical ZrO₂ balls for 15 second. The milled powder was sieved through stack of 3 sieves to classify powders into 3 different size groups of 75˜ 150 μm, 38˜75 μm, and ≤38 μm. The morphologies of as received Tosoh PX-328 powder and 3 different recycled powders with different sizes are shown in FIGS. 4A-4H.

Some of the 1 hour attrition milled powders were added with 1 wt % and 2 wt % PVA binder, and further wet ball milled for 1 hour by placing it on a roller mill. After wet ball milling for 1 hour, the powders with 1 wt % and 2 wt % PVA binder were dried at 110° C. for 1 hour. The re-agglomerated powder after drying was milled with a Spex mixer with 150 g of 2 mm diameter spherical ZrO₂ balls for 1 min followed by milling with 10 g of 1 mm diameter spherical ZrO₂ balls for 15 second. The milled powder was sieved through stack of 3 sieves to classify powders into 3 different size groups of 75˜ 150 μm, 38˜75 μm, and ≤38 μm.

All 3 different powders were attrition milled by using a Netzsch mill without binder, with 1 wt % PVA binder, and with 2 wt % PVA binder. The 3 different size groups, were pressed at pressures of 24.4 ksi, 35.8 ksi, and 57.4 ksi, and the results are summarized in Table 2. All pellets pressed without binder were delaminated. Pellets pressed with 1 wt % PVA binder showed end-capping at a pressure of 35.8 ksi and delaminated at a pressure of 57.4 ksi. However, all pellets pressed with 2 wt % PVA binder were pressed successfully without delamination at pressure of 35.8 ksi and also at a pressure of 57.4 ksi.

In this particular example, this result shows that the powder attrition milled for 1 hour by Netzsch mill need to have 2 wt % PVA binder to be pressed without delamination at pressures of 35.8 ksi and 57.4 ksi.

TABLE 2
Pressing of 3 different recycled, attrition milled powders with different
sizes, at different pressures of 24.4 ksi, 35.8 ksi and 57.4 ksi without
binder, with 1 wt % PVA binder, and with 2 wt % PVA binder addition.

| | | No binder | | 1 wt % binder | | 2 wt % binder | |
|---|---|---|---|---|---|---|---|
| | | 24.4 ksi | 35.8 ksi | 35.8 ksi | 57.4 ksi | 35.8 ksi | 57.4 ksi |
| 75~150 | μm | Delamination | Delamination | OK | x | OK | x |
| 38~75 | μm | Delamination | Delamination | Endcapping | Delamination | OK | OK |
| ≤38 | μm | Delamination | Delamination | Endcapping | x | OK | x |

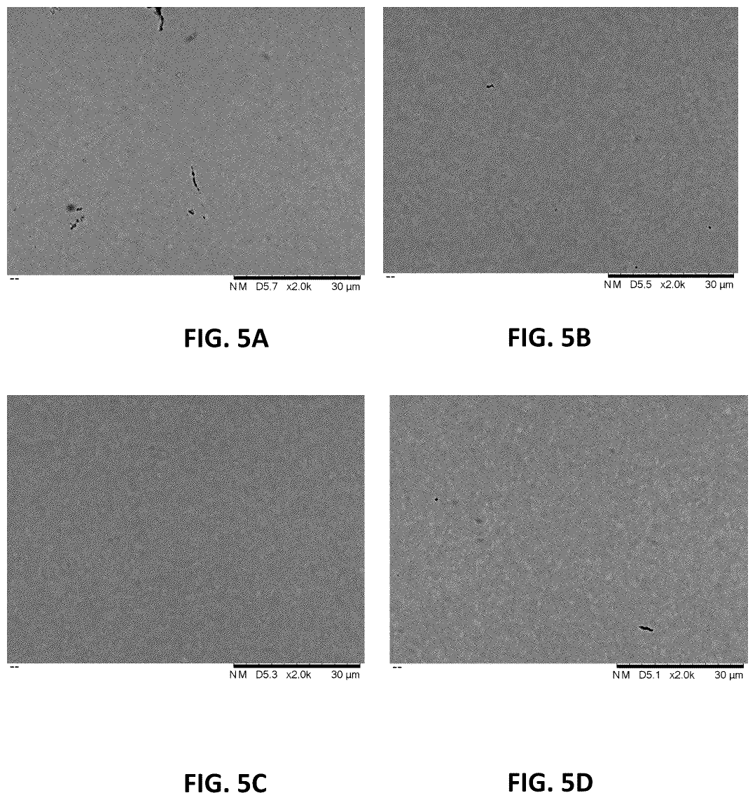

3 different powders with sizes of 75˜ 150 μm, 38˜75 μm, and smaller than 38 μm which were added with 2 wt % PVA binder after attrition milling for 1 hour were pressed into discs with diameter of 20 mm. Three discs were pressed at pressure of 35.8 ksi and one disc was pressed at pressure of 57.4 ksi. All pressed discs were sintered at 1580° C. for 2 hour 30 min, and the microstructures are represented in FIG. 5. As can be seen in FIG. 5, the microstructure showed a large number of pores.

### Example 3

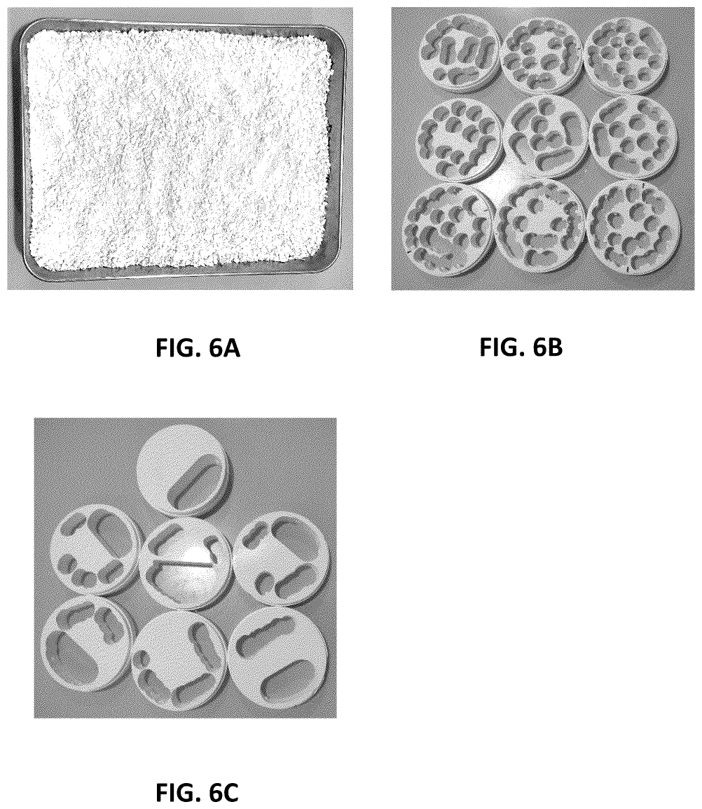

To evaluate the differences in effectiveness of milling of different types of scrap CAD/CAM ZrO₂ materials, 3 different types of scrap ZrO₂ blocks were selectively ball milled. 3 different types of scrap ZrO₂ materials are shown in FIG. 6. 3938 g of scrap ZrO₂ material pre-size reduced below 4.75 mm (4750 μm), 3938 g of blocks which have thickness of 14 mm and diameter of 98 mm and have minimal remaining ZrO₂, and 3938 g of blocks which have thickness of 20 mm and diameter of 98 mm and have larger quantity remaining ZrO₂, as shown in FIG. 6A, FIG. 6B, FIG. 6C, respectively, were wet ball milled for 10 hour by using 20 mm diameter cylindrical ZrO₂ balls.

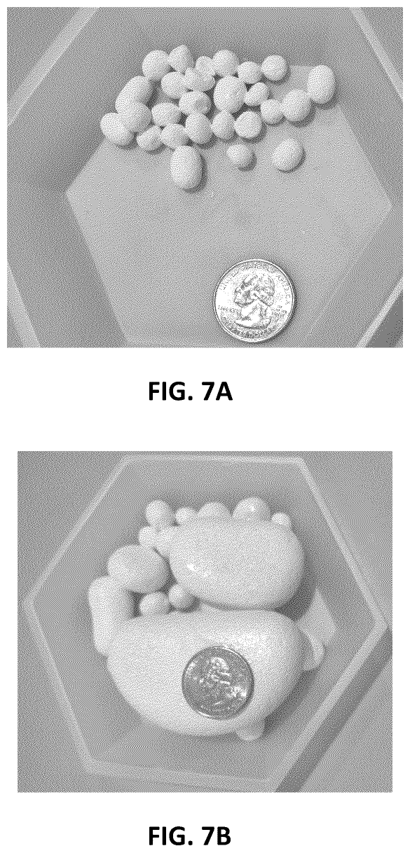

Scrap ZrO₂ material with size smaller than 4.75 mm could be milled into very fine slurry within 2 hour by ball milling using 20 mm diameter cylindrical ZrO₂ balls. After 10 hour wet ball milling, the powder was very fine, and all particles were much smaller than 100 μm. Scrap ZrO₂ blocks with thicknesses of 14 mm thickness, diameters of 98 mm diameter, and with minimal remaining ZrO₂ could be milled down to relatively finer size, but about 29 g of powders were bigger than 100 μm after 10 hour ball milling, and the picture of particles larger than 100 μm is shown in FIG. 7A. The scrap ZrO₂ blocks with thicknesses of 20 mm, with diameters of 98 mm, and with larger quantity remaining ZrO₂ could only be milled down to relatively larger size, and about 349 g of powders were bigger than 100 μm after ball milling for 10 hour, and the picture of particles larger than 100 μm is shown in FIG. 7B.

### Example 4

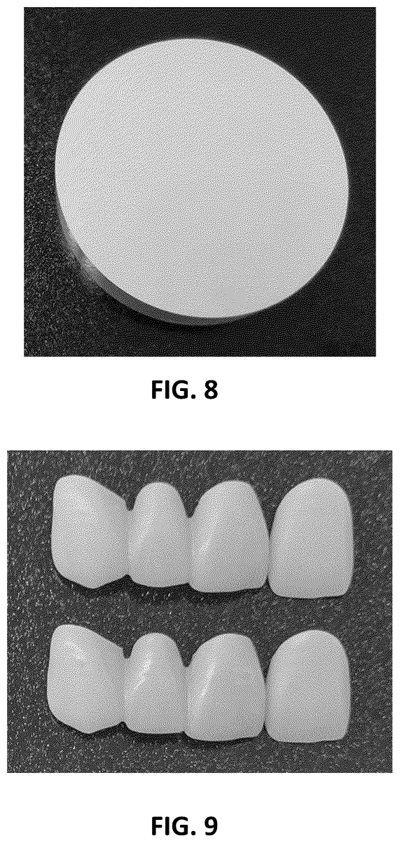

Scrap ZrO₂ blocks were mechanically size reduced, and the broken particles were passed sequentially through 3 different sieves with opening diameters of 4750, 1700, and 500 μm. All of the powders that passed through a sieve with opening diameter of 500 μm were further size reduced by using a spin mixer to pass through the sieve with opening size of 75 μm. All the powder with size smaller than 75 μm were mixed with 28 wt % of DI H₂O and 0.5 wt % (to the weight of ZrO₂ powder) of Dolopix CE-64 as a dispersant. The mixture was attrition milled by using a Netzsch mill at 2500 rpm for 1 hour. The milled powder was further wet ball milled with 2 wt % of PVA binder for 1 hour. After ball milling, the powder slurry was dried at 110° C. To break down the agglomeration of dried, re-agglomerated powder after drying, the powder was milled by using a spin mixer and then sequentially sieved using sieves with opening sizes of 75˜ 150 μm, 38˜75 μm, and ≤38 μm. The sieved powders were composed of 1.8 wt % of powder with sizes of ≤38 μm, 18.9 wt % of powder with size of 38˜75 μm, and 79.3 wt % of powder with size of 75˜ 150 μm, and all powders with different sizes were mixed together. The final mixed powder of about 8500 g was pressed into 17 blocks with approximate diameter of 102 mm by using commercial scale pressing machine at a pressure of about 24.4 ksi. The optical micrograph of the pressed block is shown in FIG. 8 and the block showed a density of about 3.22 g/cm³ (52.7% of the theoretical density).

One 3-unit bridge and one single crown were milled from commercial BruxZir Shaded 16+ A2 shade block and also from recycled BruxZir Shaded 16+ A2 shade block and were then sintered at 1580° C. for 2 hour 30 min. The optical micrographs of all sintered teeth are shown in FIG. 9. The colors of two different group of teeth did not show any significant difference.

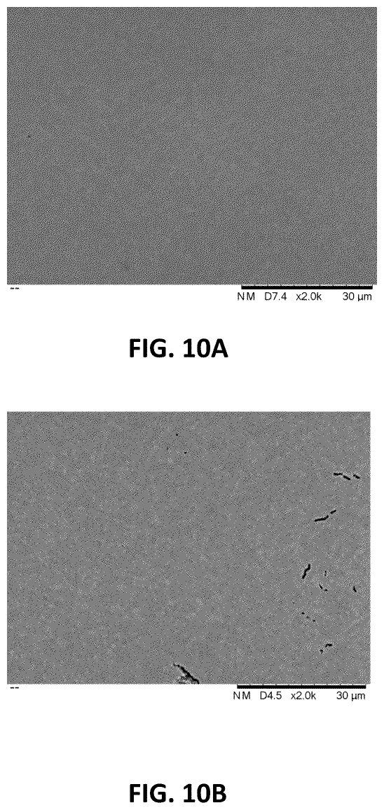

The SEM micrograph of sintered ZrO₂ block made from commercial shaded 16+ A2 shade block and that of sintered ZrO₂ block made from recycled shaded 16+ A2 shade block are shown in FIG. 10A and FIG. 10B, respectively. As can be seen in FIG. 10A, the block made from commercial ZrO₂ powder showed very dense microstructure.

However, as can be seen FIG. 10B, the block made from recycled ZrO₂ powder indicated presence of significant amounts of pores, possibly due to agglomeration of powder after drying. As can be seen in the Table 3, the sintered block made from commercial shaded 16+ powder had higher strength and translucency of 1046±120 MPa and 47%, respectively. The sintered block made from recycled shaded 16+ powder had lower strength and translucency of 509±72 MPa and 43%, respectively. Translucency measurement was made using a Konica Minolta CM-5 at 700 nm.

TABLE 3
Comparison of strengths and translucencies of sintered
block made from new BruxZir Shaded 16(+) A2 shade
ZrO₂ powder and those of sintered block made from recycled
BruxZir Shaded 16(+) A2 shade ZrO₂ powder.

| | Strength | | Translucency |
|---|---|---|---|
| | (MPa) | | (%) |
| New BruxZir Shaded 16(+) | 1046 ± 120 | MPa | 47% |
| A2 shade | | | |
| Recycled BruxZir Shaded 16(+) | 509 ± 72 | MPa | 43% |
| A2 shade | | | |

### Example 5

Two different kinds of ZrO₂ media were used in the milling of scrap ZrO₂ blocks. The first media was a spherical ball with diameter of 5 mm and the second media was a cylindrical ball with height of 20 mm and diameter of 20 mm. The weight of single 5 mm spherical ball was 0.45 g and the weight of single 20 mm cylindrical ball was 36.5 g.

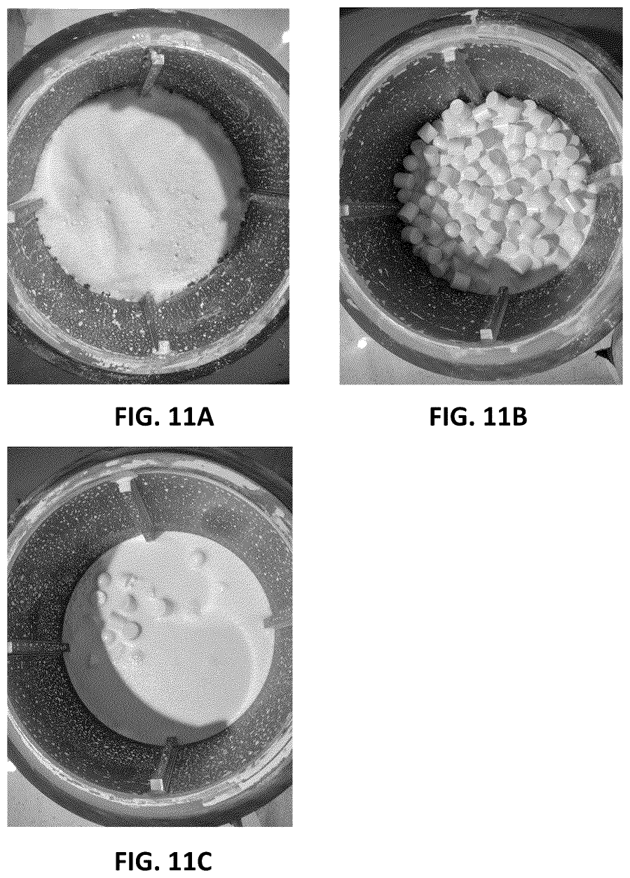

Scrap ZrO₂ blocks were mechanically size reduced, and the broken pieces were passed through #4 sieve which has opening of 4.75 mm (4750 μm). 3938 g of pre-milled powder with size smaller than 4.75 mm, 22411 g of 5 mm spherical ZrO₂ balls, 1531 g of H₂O, 19.69 g of Dolopix CE 64 were loaded into ball milling container with volume of 11078 cm³ (diameter: 24 cm and height: 24.5 cm). The ball milling was conducted with rotational speed of 75 rpm for 10 hour. After ball milling for 10 hour, as can be seen in FIG. 11A, the slurry was foamy and about 750 g of powder were larger than 3 mm.

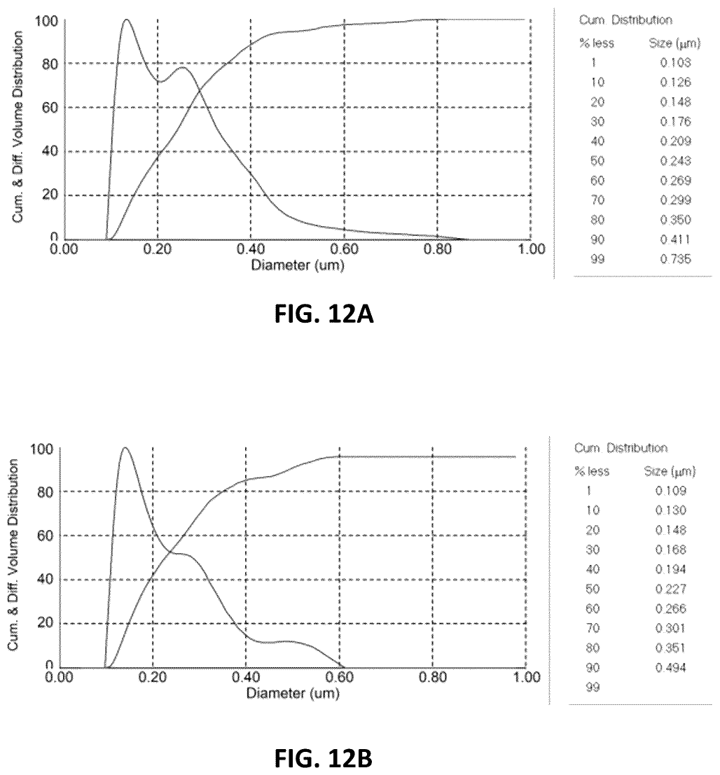

Scrap ZrO₂ blocks were mechanically size reduced and the broken pieces were passed through #4 sieve which has opening of 4.75 mm (4750 μm). 3938 g of pre-milled powder with size smaller than 4.75 mm. 22411 g of 20 mm diameter cylindrical ZrO₂ balls, 1531 g of H₂O, 19.69 g of Dolopix CE 64 were loaded into ball milling container with volume of 11078 cm³ (diameter: 24 cm and height: 24.5 cm). The ball milling was conducted with rotational speed of 75 rpm for 10 hour. During ball milling for 10 hour, the milling was stopped and checked inside of ball milling chamber after 1 hour, 2 hour, 3 hour, 5 hour, 7 hour, and 10 hour. Even after ball milling for 2 hour, the powder was milled into very fine size, and very fine slurry was formed inside of ball milling chamber. FIG. 11B shows fine slurry formation inside of ball milling chamber after 10 hour wet ball milling. As can be seen in FIG. 12A, the D₍₅₀₎ of the particle after hour ball milling was about 0.248 μm. After 10 hour of ball milling, 5 blocks with diameter of 102 mm were casted, but all 5 blocks showed formation of cracks.

Scrap ZrO₂ blocks were mechanically size reduced, and the broken pieces were passed through #4 sieve which has opening of 4.75 mm (4750 μm). 3938 g of pre-milled powder with size smaller than 4.75 mm, 11205.5 g of 5 mm diameter spherical ZrO₂ balls, 11205.5 g of 20 mm diameter cylindrical ZrO₂ balls, 1531 g of H₂O, 19.69 g of Dolopix CE 64 were loaded into ball milling container with volume of 11078 cm³ (diameter: 24 cm and height: 24.5 cm). The ball milling was conducted with rotational speed of 75 rpm for 10 hour. During ball milling for 10 hour, the milling was stopped and checked inside of ball milling chamber after 1 hour, 2 hour, 3 hour, 5 hour, 7 hour, and 10 hour. Even after ball milling for 2 hour, the powder was milled into very fine size, and very fine slurry was formed inside of ball milling chamber. FIG. 11C shows fine slurry formation inside of ball milling chamber after 10 hour wet ball milling. As can be seen in FIG. 12B, the D₍₅₀₎ of the particles after 10 hour ball milling was about 0.227 μm. After 10 hour ball milling, 5 blocks with diameter of 102 mm were casted, and all 5 blocks showed no formation of cracks after drying and bisquing.

### Example 6

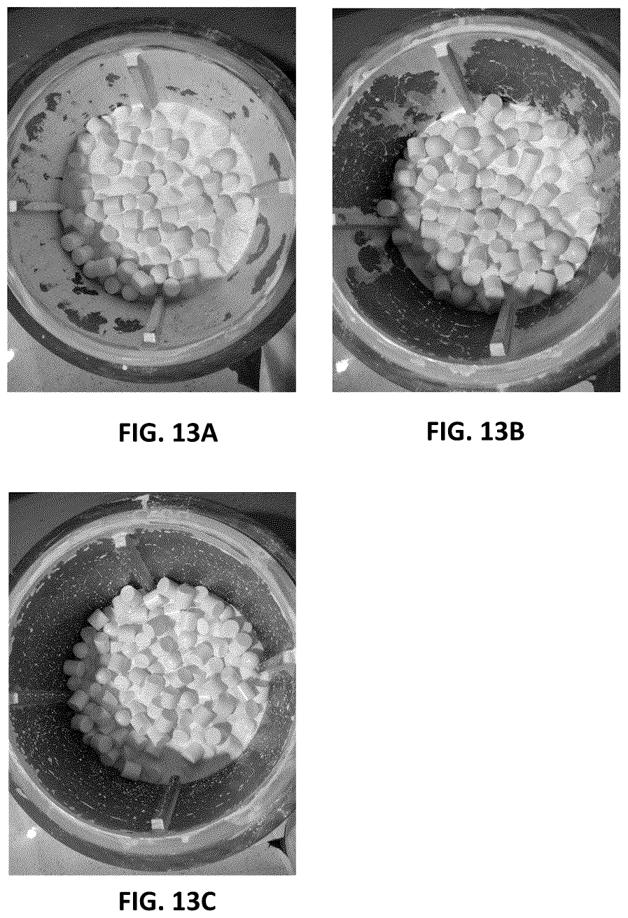

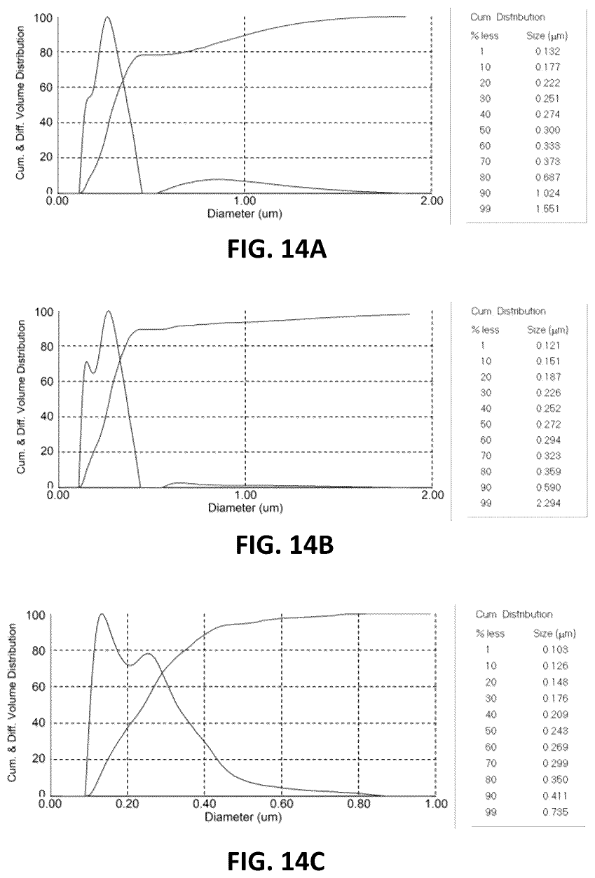

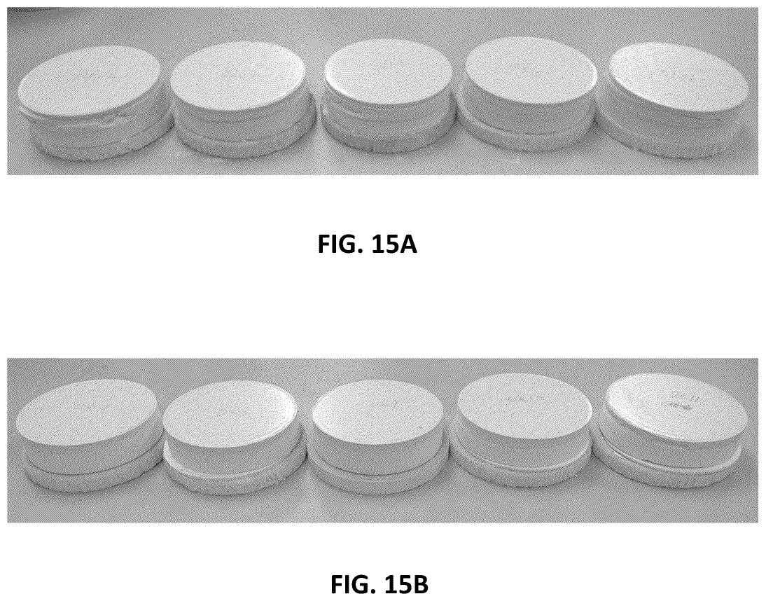

Scrap ZrO₂ blocks were mechanically pre-milled and the broken pieces were passed through #4 sieve which has opening of 4.75 mm (4750 μm). 3938 g of pre-milled powder with size smaller than 4.75 mm, 22411 g of 20 mm diameter cylindrical ZrO₂ balls, 1531 g of H₂O, 19.69 g of Dolopix CE 64 were loaded into ball milling container with volume of 11078 cm³ (diameter: 24 cm and height: 24.5 cm). The ball milling was conducted with rotational speed of 75 rpm for 3 hour. During ball milling for 3 hour, the milling was stopped and checked inside of ball milling chamber after 1 hour, 2 hour, and 3 hour. Even after ball milling for 2 hour, the powder was milled into very fine size and very fine slurry was formed inside of ball milling chamber. FIG. 13A shows fine slurry formation inside of ball milling chamber after 3 hour wet ball milling. As can be seen in FIG. 14A, the D₍₅₀₎ of the particles after 3 hour ball milling was about 0.300 μm. After 3 hour ball milling, 5 blocks with diameter of 102 mm were casted, but as can be seen in FIG. 15A all 5 blocks showed formation of cracks.

Scrap ZrO₂ blocks were mechanically pre-milled and the broken pieces were passed through #4 sieve which has opening of 4.75 mm (4750 μm). 3938 g of pre-milled powder with size smaller than 4.75 mm, 22411 g of 20 mm diameter cylindrical ZrO₂ balls, 1531 g of H₂O, 19.69 g of Dolopix CE 64 were loaded into ball milling container with volume of 11078 cm³ (diameter: 24 cm and height: 24.5 cm). The ball milling was conducted with rotational speed of 75 rpm for 5 hour. During ball milling for 5 hour, the milling was stopped and checked inside of ball milling chamber after 1 hour, 2 hour, 3 hour, and 5 hour. Even after ball milling for 2 hour, the powder was milled into very fine size and very fine slurry was formed inside of ball milling chamber. FIG. 13B shows fine slurry formation inside of ball milling chamber after 5 hour wet ball milling. As can be seen in FIG. 14B, the D₍₅₀₎ of the particles after 5 hour ball milling was about 0.272 μm. After 5 hour ball milling, 5 blocks with diameter of 102 mm were casted, but as can be seen in FIG. 15B 3 blocks out of 5 blocks showed formation of cracks.

Scrap ZrO₂ blocks were mechanically pre-milled and the broken pieces were passed through #4 sieve which has opening of 4.75 mm (4750 μm). 3938 g of pre-milled powder with size smaller than 4.75 mm, 22411 g of 20 mm diameter cylindrical ZrO₂ balls, 1531 g of H₂O, 19.69 g of Dolopix CE 64 were loaded into ball milling container with volume of 11078 cm³ (diameter: 24 cm and height: 24.5 cm). The ball milling was conducted with rotational speed of 75 rpm for 10 hour. During the operation of ball milling for 10 hour, the milling was stopped and checked inside of ball milling chamber after 1 hour, 2 hour, 3 hour, 5 hour, 7 hour, and 10 hour. Even after ball milling for 2 hour, the powder was milled into very fine size and very fine slurry was formed inside of ball milling chamber. FIG. 13C shows fine slurry formation inside of ball milling chamber after 10 hour wet ball milling. As can be seen inf FIG. 14C, the D₍₅₀₎ of the particles after 10 hour ball milling was about 0.248 μm. After 10 hour ball milling, 5 blocks with diameter of 102 mm were casted, and all 5 blocks showed no cracks.

### Example 7

Scrap ZrO₂ powder was wet ball milled and also dry ball milled by using cylindrical balls with diameter of 20 mm and height of 20 mm. Scrap ZrO₂ blocks were mechanically pre-milled and the broken pieces were passed through #4 sieve which has opening of 4.75 mm (4750 μm).

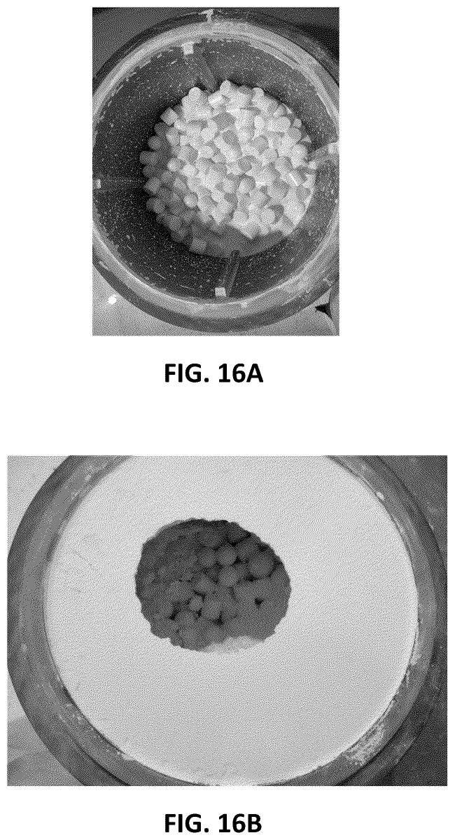

At first, 3938 g of pre-milled powder with size smaller than 4.75 mm, 22411 g of 20 mm diameter cylindrical ZrO₂ balls, 1531 g of H₂O, 19.69 g of Dolopix CE 64 were loaded into ball milling container with volume of 11078 cm³ (diameter: 24 cm and height: 24.5 cm). The wet ball milling was conducted with rotational speed of 75 rpm for 10 hour. During the operation of ball milling for 10 hour, the milling was stopped and checked inside of ball milling chamber after 1 hour, 2 hour, 3 hour, 5 hour, 7 hour, and 10 hour. Even after ball milling for 2 hour, the powder was milled into very fine size and very fine slurry was formed inside of ball milling chamber. As shown in FIG. 16A, very fine slurry was formed after wet ball milling for 10 hour. After 10 hour ball milling, 5 blocks with diameter of 102 mm were casted, and all 5 blocks showed no formation of cracks after drying and bisquing.

Secondly, 3938 g of pre-milled powder with size smaller than 4.75 mm, 22411 g of 20 mm diameter cylindrical ZrO₂ balls, and 78.76 g of corn starch were loaded into ball milling container with volume of 11078 cm³ (diameter: 24 cm and height: 24.5 cm). The dry ball milling was conducted with rotational speed of 75 rpm for 10 hour. During the operation of dry ball milling for 10 hour, the milling was stopped and checked inside of ball milling chamber after 1 hour, 2 hour, 3 hour, 5 hour, 7 hour, and hour. Even after ball milling for 10 hour, the powder showed very fine particle size, but as can be seen in FIG. 16B, lots of powder were agglomerated and stuck at the top.

### Example 8

Scrap ZrO₂ powders were wet ball milled and also dry ball milled by using mixture of spherical balls with diameter of 5 mm and cylindrical balls with diameter of 20 mm and height of 20 mm. Scrap ZrO₂ blocks were mechanically pre-milled and the broken pieces were passed through #4 sieve which has opening of 4.75 mm (4750 μm).

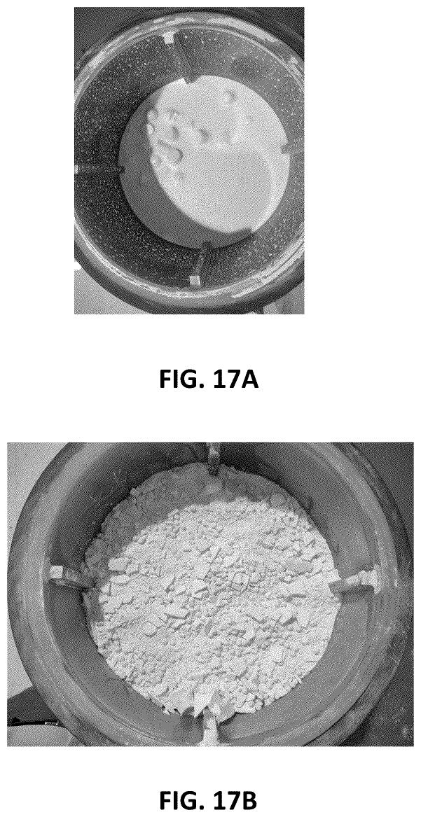

At first, 3938 g of pre-milled powder with size smaller than 4.75 mm, 11205.5 g of 5 mm diameter spherical ZrO₂ balls, 11205.5 g of 20 mm diameter cylindrical ZrO₂ balls, 1531 g of H₂O, 19.69 g of Dolopix CE 64 were loaded into ball milling container with volume of 11078 cm³ (diameter: 24 cm and height: 24.5 cm). The wet ball milling was conducted with rotational speed of 75 rpm for 10 hour. During wet ball milling for 10 hour, the milling was stopped and checked inside of ball milling chamber after 1 hour, 2 hour, 3 hour, 5 hour, 7 hour, and 10 hour. Even after ball milling for 2 hour, the powder was milled into very fine size and very fine slurry was formed inside of ball milling chamber. As shown in FIG. 17A, very fine slurry was formed after wet ball milling for 10 hour.

After 10 hour ball milling, 5 blocks with diameter of 102 mm were casted, and all 5 blocks showed no formation of cracks after drying and bisquing.

Secondly, 3938 g of pre milled powder with size smaller than 4.75 mm, 11205.5 g of 5 mm diameter spherical ZrO₂ balls, 11205.5 g of 20 mm diameter cylindrical ZrO₂ balls, and 78.76 g of corn starch were loaded into ball milling container with volume of 11078 cm³ (diameter: 24 cm and height: 24.5 cm). The dry ball milling was conducted with rotational speed of 75 rpm for 10 hour. During dry ball milling for 10 hour, the milling was stopped and checked inside of ball milling chamber after 1 hour, 2 hour, 3 hour, 5 hour, 7 hour, and 10 hour. Even after ball milling for 10 hour, as can be seen in FIG. 17B, the powders showed agglomeration and diameters of some particles were larger than 1 mm.

### Example 9

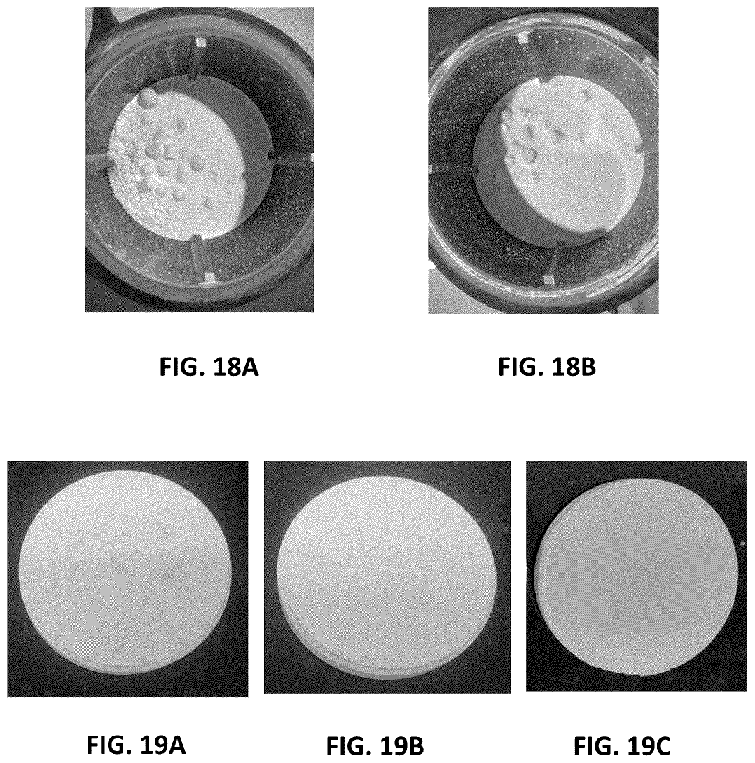

3938 g of pre-milled powder with size smaller than 4.75 mm, 11205.5 g of 5 mm diameter spherical ZrO₂ balls, 11205.5 g of 20 mm diameter cylindrical ZrO₂ balls, 1531 g of H₂O, 19.69 g of Dolopix CE 64 were loaded into ball milling container with volume of 11078 cm³ (diameter: 24 cm and height: 24.5 cm). The wet ball milling was conducted with rotational speed of 75 rpm either for 5 hour or for 10 hour. After ball milling for 5 hour or 10 hour, the ZrO₂ slurry showed very fine particles, as shown in FIGS. 18A-18B.

Three different types of ZrO₂ blocks were prepared after milling. The first type of blocks was manufactured after ball milling of scrap ZrO₂ blocks for 5 hour and then slip casting. The second type of blocks was prepared after ball milling of scrap ZrO₂ materials for 10 hour and then slip casting. As can be seen in FIGS. 19A-19C, the ZrO₂ block made from the scrap ZrO₂ powder after 5 hour wet milling followed by casting showed large voids, but the blocks made from the scrap ZrO₂ powder after 10 hour wet milling followed by casting did now show any voids.

3938 g of scrap ZrO₂ blocks were ball milled for 10 hour by using 20 mm diameter spherical ZrO₂ balls with addition of 1531 g of DI water and 19.69 g of Dolopix CE 64 as a dispersant. 4944 g of the ball milled ZrO₂ slurry was further attrition milled by Netzsch milling at 2000 rpm for 1 hour. The third type of blocks was obtained after slip casting the attrition milled slurry. As can be seen in FIG. 19C, the block made from ball milling for 10 hour, attrition milling for 1 hour, and then casting did not show any voids.

Two different types of ZrO₂ blocks, the one obtained after ball milling for 10 hour followed by casting, and the other one made after ball milling for 10 hour, attrition milling for 1 hour, and then casting were sintered at 1580° C. for 1 hour 30 min. The strengths, toughnesses, and translucencies of those two blocks were accordingly compared to those of BruxZir Shaded 16(+) A2 shade block made from new powder, and the results are summarized in Table 4. As can be seen in Table 4, the block made by hour ball milling and then casting had similar strength to that of block made from new powder, but had lower translucency of 43% compared to 47% of block made from new powder. The strength, toughness, and translucency of the block made by 10 hour ball milling, 1 hour attrition milling, and then casting were accordingly similar to those of block made from new powder.

TABLE 4
Comparison of strength, toughness, and translucency
of new BruxZir Shaded 16(+) A2 shade and those of
recycled BruxZir Shaded 16(+) A2 shade.

| | Strength | Toughness | Translucency |
|---|---|---|---|
| | (MPa) | (MPa · m^{1/2}) | (%) |
| New BruxZir Shaded 16(+) | 1046 ± 140 | 4.76 ± 0.04 | 47% |
| A2 shade block | | | |
| Recycled BruxZir Shaded | 1141 ± 83 | 4.68 ± 0.10 | 43% |
| 16(+) A2 shade block (10 | | | |
| hour ball milling → Casting) | | | |
| Recycled BruxZir Shaded | 1055 ± 120 | 4.61 ± 0.08 | 49% |
| 16(+) A2 shade block (10 | | | |
| hour ball milling→ 1 hr | | | |
| attrition milling → Casting) | | | |

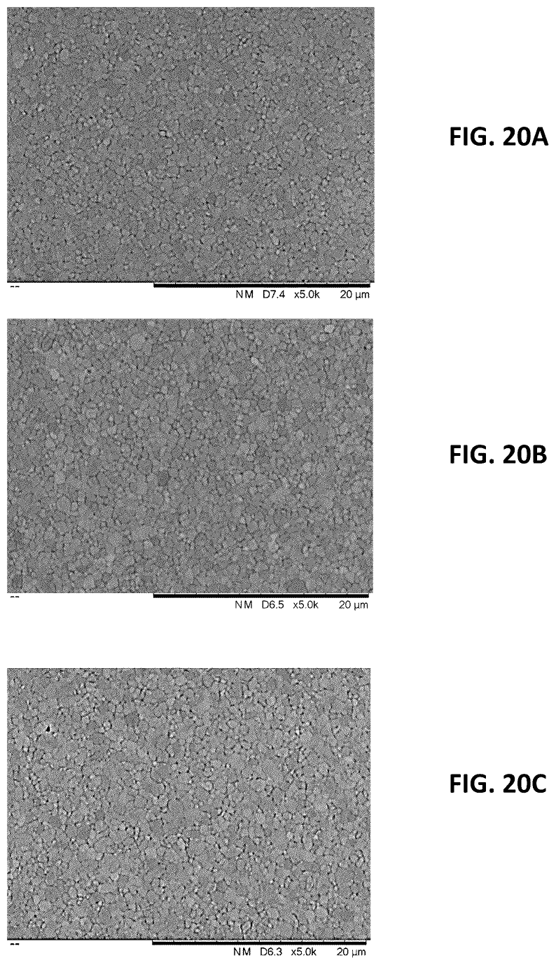

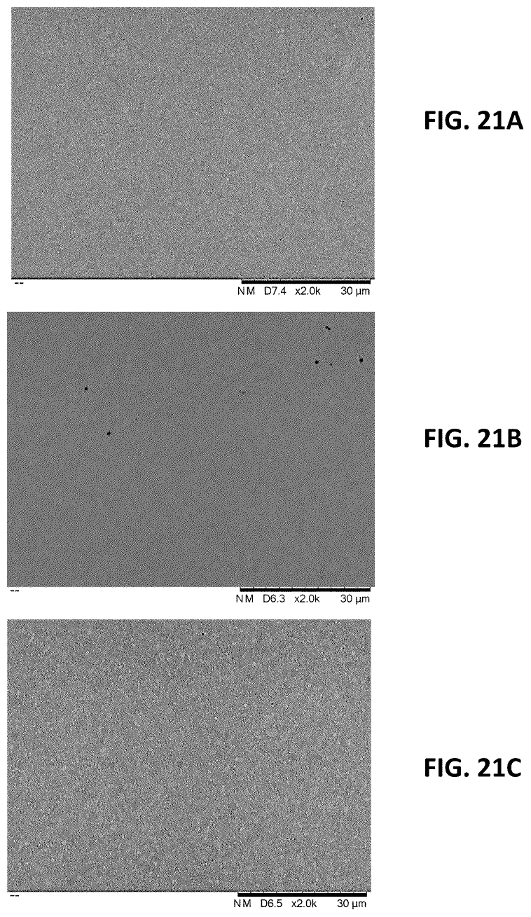

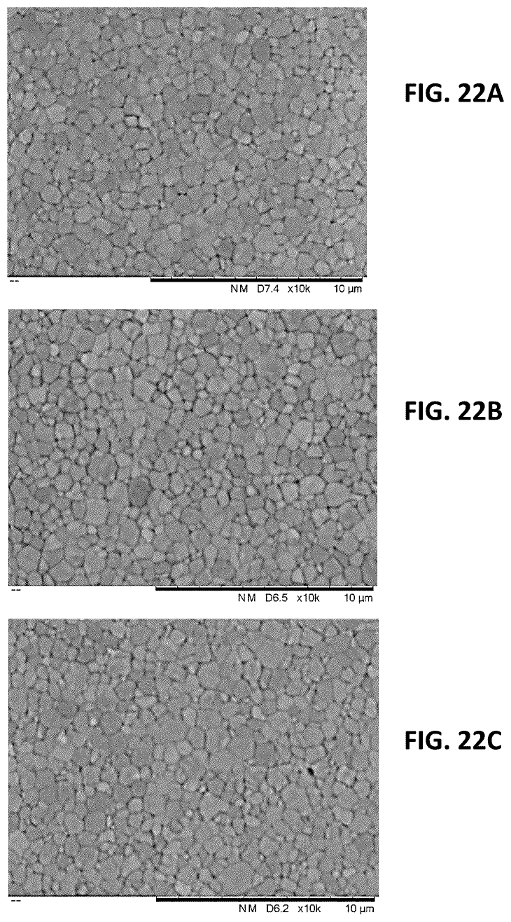

As can be seen in FIGS. 20A-20C, FIGS. 21A-21C, FIGS. 22A-22C, the microstructures of block made from the scrap ZrO₂ power ball milled for 10 hour followed by casting showed more pores than those of block made from new powder. However, the microstructure of the block made from the powder ball milled for 10 hour, attrition milled for 1 hour, and then casted was very similar grain size and also as dense as that of the block made from new powder.

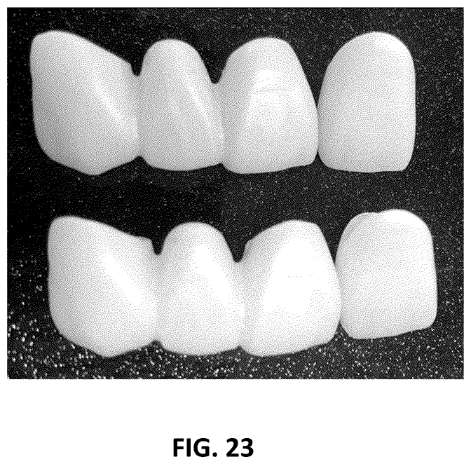

One 3-unit bridge and one single crown were milled from commercial BruxZir Shaded 16(+) A2 shade block and also from recycled BruxZir Shaded 16(+) A2 shade block made from the powder ball milled for 10 hour, attrition milled for 1 hour, and then casted were sintered them at 1580° C. for 2 hour 30 min. The optical micrographs of all sintered teeth are shown in FIG. 23. The colors of two different group of teeth did not show any significant difference.

### Example 10

Scrap zirconia powder with diameter of less than 100 mm was further size reduced to a size of less than 5 mm using a Jaw crusher with zirconia lined jaws. The jaw crushed material was then roll crushed to a size of less than −50 mesh using a zirconia lined roll crusher. The resulting −50 mesh material was then further milled in an Attritor mill for 14 hours with zirconia media of size 5 mm, water, and dispersant to create a slurry of zirconia particles having a D₍₅₀₎ of 0.21 μm. The solids loading of the slurry was greater than or equal to 69 wt %. Particle size was measured using an X-Ray Disc Centrifuge Particle Size Analyzer by Brookhaven Instruments.

### Test Methods

### Fracture Toughness Test

Samples for fracture toughness testing were milled and sintered. Tabs of ceramic materials were milled out of a bisque block. The bisque tabs have the dimensions of approximately 13-15 mm, thickness=1-5 mm after sintering. Polishing was carried out to obtain a scratch-free surface according to the polishing steps of Table 5. The polished side with the fewest observed defects was chosen as the side for fracture toughness test. Fracture toughness testing was performed on a Shimadzu Micro Hardness Tester (HMV-G21) testing machine with a Vickers indenter fixture. The length of the crack and indentation diagonal were measured by built in optical microscope (×10, ×40). The method of testing fracture toughness was based on Brian Lawn's calculation (1980) and G. R. Anstis (J. Am. Ceram. Soc., 64(9), P533-538, 1981). Using the equation:

$${K}_{IC} = 0.0205 * [ 2 √ ( E / H ) ] * [ P / ( 3 / 2 √ C ) ]$$

Wherein K_{ic}: Fracture Toughness (MPa·m^{-1/2}); E: Young's modulus (GPa); H: Vickers hardness (GPa)*, calculated by

$$H = 1.854 * ( P / {d}^{2} )$$

Wherein P applied load (N); C: crack length from the center of the impression to the crack tip; d: the length of the indentation diagonal. (Hardness Measurement Ref.: Vander, G. F. (2000). Microindentation hardness testing according to H. Kuhn & D. Medlin (Eds.), ASM Handbook, Volume 8: Mechanical Testing and Evaluation (pp. 221-231). ASM International.)

TABLE 5

| | Grinding | | Force | Head | Plate | Duration |
|---|---|---|---|---|---|---|
| Step # | Grit | Polishing Media | (lbs) | Speed(rpm) | Speed (rpm) | (min) |
| 1 | 80 | — | 30 | 80 | 120 | 0.5-1 |
| 2 | 220 | — | 30 | 80 | 120 | 1 |
| 3 | 500 | — | 30 | 80 | 120 | 7 |
| 4 | 1200 | — | 30 | 80 | 120 | 12 |
| 5 | — | 15 um diamond suspension | 30 | 80 | 120 | 15 |
| 6 | — | 3 um diamond suspension | 30 | 80 | 120 | 15 |
| 7 | — | 1 diamond suspension | 30 | 80 | 120 | 15 |
| 8 | — | 0.06 SiO₂ Silica Suspension | 30 | 80 | 120 | 15 |

### Translucency

Sintered body translucency was determined by measuring the percent transmittance of D65 light at a wavelength of 700 nm from a 1 to 1.1 mm thick sintered sample. Translucency wafers were sectioned or milled from a bisque block and machined to a diameter corresponding to a final diameter of approximately 30 mm after sinter. The wafers were then ground flat until visually free of defects with 1200 grit and 2000 grit SiC polishing paper. The final bisque thickness corresponded to 1 mm after sintering and polishing. Samples ground to the desired shape were removed of surface dust and then sintered according to the sintering profile(s) described herein.

Total transmittance spectra were measured between the wavelengths of 360 nm to 740 nm with a Konica-Minolta CM5 spectrophotometer illuminated by a D65 light source for all samples. Information contained in the data tables herein refer to measurements at 700 nm or 500 nm wavelengths, as indicated, which are extracted from these measurements. The spectrophotometer was calibrated to white and black prior to measurement. Translucency samples were placed flush against the (approximately) 25 mm integrating sphere aperture. A minimum of two spectra were collected per sample and average to yield a final measured transmittance spectra (S-TM). Collected transmittance data may be reported as “percent (%) transmittance”.

In view of the many possible embodiments to which the principles of the disclosed invention may be applied, it should be recognized that the illustrated embodiments are only preferred examples and should not be taken as limiting the scope of the invention.

What is claimed is:

1. A method, comprising:
collecting scrap ZrO₂ material;
reducing sizes of the scrap ZrO₂ material to produce a reduced-size scrap ZrO₂ material;
preparing a ceramic slurry comprising the reduced-size scrap ZrO₂ material, deionized water, and at least one dispersant;
milling the ceramic slurry to produce a milled ZrO₂ slurry, wherein the milled ZrO₂ slurry comprises a ZrO₂ powder with a particle size distribution D₍₅₀₎ of 0.05 μm to 1.0 μm; and
casting the milled ZrO₂ slurry to prepare a ceramic block.

2. The method of claim 1, further comprising sieving the reduced-size scrap ZrO₂ material.

3. The method of claim 1, wherein the reduced-size ZrO₂ material comprises ZrO₂ particles with a particle size distribution D₍₅₀₎ of 1 mm to 10 mm.

4. The method of claim 2, wherein the sieved, reduced-size ZrO₂ material comprises ZrO₂ particles with a particle size of 0.0013 mm to 8 mm.

5. The method of claim 1, wherein reducing sizes of the scrap ZrO₂ material comprises milling the scrap ZrO₂ material using a mechanical milling process.

6. The method of claim 1, wherein the milling of the ceramic slurry comprises wet ball milling.

7. The method of claim 6, wherein the wet ball milling comprises using a mixture of 5 mm and 20 mm diameter balls.

8. The method of claim 6, wherein the wet ball milling comprises using 5 mm diameter balls.

9. The method of claim 6, wherein the wet ball milling is performed for 10 hours.

10. The method of claim 1, wherein reducing sizes of the scrap ZrO₂ material comprises roll crushing, and the milling of the ceramic slurry comprises wet milling or dry milling.

11. The method of claim 1, wherein the milled ZrO₂ slurry comprises a ZrO₂ powder with a particle size distribution D₍₅₀₎ of 0.1 μm to 0.5 μm.

12. The method of claim 1, further comprising sintering the ceramic block.

13. The method of claim 1, further comprising adding at least one binder to the ceramic slurry.

14. The method of claim 1, further comprising attrition milling the milled ZrO₂ slurry.

15. The method of claim 14, wherein the attrition milling is performed for one hour.

16. The method of claim 1, wherein the scrap ZrO₂ material is produced during a process for making a dental prosthetic device.

17. The method of claim 12, wherein the sintered ceramic block has a flexural strength of 700 to 1500 MPa, a fracture toughness of 1 to 8 MPa·m^{1/2}; and a transmittance of 30% to 65% at 700 nm (when measured on a 1 mm thick fully sintered ceramic body).

18. The method of claim 12, wherein the sintered ceramic block has a flexural strength of 900 to 1200 MPa, a fracture toughness of 3 to 5 MPa·m^{1/2}; and a transmittance of 40% to 55% at 700 nm (when measured on a 1 mm thick fully sintered ceramic body).

19. The method of claim 12, further comprising forming a dental prosthetic device from the sintered ceramic block.

20. The method of claim 1, wherein the ceramic block has a size of 90 mm to 115 mm.
